(19) United States
(12) Patent Application Publication
(10) Pub. No.: US 2004/0147878 A1
(43) Pub. Date: Jul. 29, 2004

(54) Holder and transporter for fluid collecting tubes

(76) Inventors: Christopher Lyon, Euclid, OH (US); David Yarmesch, Brunswick, OH (US)

Correspondence Address: Fay, Sharpe, Fagan,, Minnich & McKee, LLP, 7th Floor, 1100 Superior Avenue, Cleveland, OH, 44114-2579, US

(21) Appl. No.: 10/753,581

(22) Filed: Jan. 8, 2004

Related U.S. Application Data
(60) Provisional application No. 60/439,203, filed on Jan. 10, 2003.

(51) Int. Cl. A47B 73/00; A61M 5/32

(52) U.S. Cl. 604/192; 211/74

(57) ABSTRACT

A sample collection method and apparatus for holding and transporting tubes is provided which includes a tube holder having an elongated body with at least one fluid collection sleeve thereon for receiving and retaining at least one fluid collection tube. The apparatus includes at least one sample retriever sleeve thereon for receiving and retaining a sample retriever tube.

This application claims priority of Provisional Application Serial No. 60/439,203 filed Jan. 10, 2003.

### BACKGROUND OF THE INVENTION

This invention relates to a holding, safety, and transporting device, and method, for collecting fluids for laboratory testing and, more particularly, to a device for securing undiluted and substantially uncontaminated blood samples for laboratory testing.

The use of hypodermic needles in collecting fluids, particularly collecting multiple blood samples in evacuated tubes from a donor or patient presents a cumbersome and dangerous condition with respect to accidental needle sticks (or needle nicks) to anyone exposed to such needles, and/or the accidental mishandling of the blood samples. This is especially true for the technician or drawer collecting the fluid or blood sample. The situation is especially aggravated when the individual is a paramedic (or other health care worker, i.e. nurse, emergency room technician, etc.) and the multiple blood samples are being collected from the donor in a moving vehicle and/or in an emergency situation.

After collecting a sample, the hypodermic needle and blood sample can be contaminated by a variety of disease-causing agents such as, but not limited to, hepatitis B virus or HIV, the virus which leads to acquired immune deficiency syndrome (AIDS). These and other diseases can be transmitted to any person who is stuck, nicked, or poked by a disease-contaminated needle and/or exposed to a mishandled sample. Due to the hectic conditions often present in emergency as well as hospital settings, one or more needles and/or one or more collection tubes can often be found lying about in the presence of patients, ambulance personnel, hospital personnel, et al. A mishandled or destroyed blood sample results in additional time or in an additional needle stick to the donor/patient in order to recollect a blood sample. Furthermore, all health care personnel accidentally stuck or nicked by a previously used needle must go through a series of blood tests in order to ascertain whether they have been infected. Therefore, it is desirable to provide a device for managing the manual handling of the collection tube containers, as well as to minimize the dangers associated with needle stick injuries by minimizing exposure to contaminated needles.

Often, mishandling of the blood samples and/or needle stick injuries occur when a paramedic, nurse, phlebotomist, medical technician, etc. (collectively referred to as “drawer”) attempts to collect multiple samples of blood in separate collection tubes such as, but not limited to, a VACUTAINER® tube. VACUTAINER® is a federally registered trademark of Becton Dickinson Company. VACUTAINER® tubes are commonly used for collecting blood samples. VACUTAINER® tubes are evacuated to facilitate “pulling” in an amount of liquid or blood for subsequent testing. The tubes can be closed with a needle-penetrable stopper that is pierceable with a hypodermic needle to facilitate “pulling” out the proper amount of liquid (or blood) for the particular test. The needle penetrable stopper seals the VACUTAINER® tube upon withdrawal of the hypodermic needle. VACUTAINER® tubes or evacuated collection tubes provide the pressure differential necessary to facilitate flow and collection of the blood. Multiple blood samples are often necessary when a variety of tests are to be run or when confirmation tests are required. The typical protocol for emergency patients en route to a hospital facility for treatment routinely requires five to six separate blood collection tubes to be filled with blood samples. To eliminate patient discomfort from multiple needle sticks, it is desirable to collect a sufficient amount of blood, which can supply all the individual VACUTAINER® tubes, with a single insertion of a hypodermic or phlebotomy needle into the vein of a donor.

VACUTAINER® tubes are used to collect the blood samples and prevent the blood samples from becoming contaminated. Typically, when blood is collected directly from the patient or blood donor, some type of flexible tubing or blood sample retriever having a double-ended needle cannula or two juxtaposed hollow needles attached thereto is utilized. Located at one end of this tubing/retriever is the intravenous (IV) end of the needle, which is inserted into the vein of a patient or donor to draw blood. At a second end is a depositing needle used to penetrate the stopper for establishing fluid communication and depositing blood in the VACUTAINER® tubes. The pressure differential causes blood to flow into the tube. When the blood has reached a preselected level in the tube, the tube is withdrawn from the depositing needle. The depositing needle must be withdrawn and inserted into multiple numbers of stopper sealed tubes in order to collect the requisite number of samples of blood. The blood sampling tubes may contain one of a variety of preparations to facilitate a particular analytical procedure. Such preparations may include, for example, preservatives, anti-coagulants, and the like. This repeated transfer of the depositing needle between the separate fluid collection tubes creates multiple opportunities for needle stick injuries to occur to the drawer. This procedure also creates multiple opportunities for mishandling, i.e. dropping, each individual and unsecured VACUTAINER® tube. Therefore, it is desirable to improve the handling and transporting of the individual VACUTAINER® tubes as well as to minimize the danger of spreading diseases from the patient to the drawer by precluding the incidents of needle stick injuries and mishandling of tubes during blood sample collection.

Various patents have issued for devices which protect medical professionals from needle stick injuries or provide a mechanism for holding multiple VACUTAINER® tubes. For example, U.S. Pat. No. 4,982,850 to Mears discloses a test tube holder with a safety shield. Another patent, U.S. Pat. No. 5,951,524 to Enriquez discloses a guard and holder for various sized VACUTAINER® tubes which provides a circular rigid plate capable of protecting the user's hand from needle sticks.

There remains, however, a need for an improved holder, transporter, identifier, and safety device.

### SUMMARY AND OBJECTS OF THE INVENTION

The present invention provides an improvement for holding and transporting tubes of the type described above which overcome the above referred to difficulties and others, and is easy to manipulate, orient, store and use. More particularly in this respect, an apparatus for holding and transporting tubes is provided which includes a tube holder having an elongated body with at least one fluid collection sleeve thereon for receiving and retaining at least one fluid collection tube. At least one sample retriever sleeve can be included on the body for receiving and retaining a sample retriever tube. In one embodiment of the invention, the device is flexible and small enough that a drawer can carry the device in a pocket and/or store multiple devices in a small compartment. In another and/or additional embodiment of the invention, the device is inexpensive enough to be a throw-away item, but can be designed for reuse.

In another aspect of the invention, an apparatus for holding and transporting tubes is provided which includes a tube holder having at least one fluid collection sleeve thereon for receiving and retaining at least one fluid collection tube. The tube holder includes a first member and a second member for retaining a securing band therebetween.

In yet another aspect of the invention, an apparatus for holding and transporting tubes is provided which includes a tube holder having at least one fluid collection sleeve thereon. A protective guard can be connected to a side of the holder and includes at least one fluid collection opening therethrough. The at least one fluid collection opening is aligned with the at least one fluid collection sleeve for receiving and retaining at least one fluid collection tube.

In still another aspect of the invention, a method of collecting fluid samples from a donor is provided which includes the following steps: mounting a tube holder to a donor's arm, the tube holder includes at least one fluid collection sleeve thereon for receiving and retaining at least one fluid collection tube, the tube holder is moveable about the arm; moving the at least one fluid collection tube toward a depositing needle, the needle is in communication with the fluid sample; filling the at least one fluid collection tube with the fluid sample; and, moving the at least one fluid collection tube away from the depositing needle.

It is, therefore, an object of the present invention to provide a holding and transporting device which secures multiple VACUTAINER® tubes during the process of taking a blood sample or multiple blood samples from a patient.

It is another object of the present invention to provide a device which allows the drawer to physically manipulate the fluid collection tubes while the tubes are in their respective sleeves. Because the drawer can hold part of the restrained collection tube, the tube is held steady while the drawer attempts insertion and withdrawal of the needle.

It is still another object of the present invention to provide a device that can be mounted upon a patient's arm during blood collection in order to allow the drawer to use both hands to facilitate the blood sample collection process.

It is yet another object of the present invention to provide a device capable of being secured around a drawer's hand whereby the collection tubes are proximal to the back of one hand without impeding use of both hands to facilitate the blood sample collection process.

It is a further object of the present invention to provide a device which is capable of securing multiple VACUTAINER® tubes, as well as a needleless attachment, a needle system, and/or any unused medications.

It is another object of the present invention to provide a protective guard surrounding one end of the VACUTAINER® tubes which protects the user's hand from needle stick injuries during the collection process. In one embodiment of the invention, the surface of the guard is configured to inhibit or prevent the needle from slipping off the guard.

It is still a further object of the present invention to provide a device which is capable of holding multiple VACUTAINER® tubes of varying sizes.

It is yet another object of the present invention to provide a device which can be attached to an IV pole.

It is still yet another object of the present invention to provide a device which can be attached to an IV bag.

It is yet another object of the present invention to provide a device capable of being rotatably secured to a blood donor whereby the device can be rotated about a donor's arm thereby providing ease of collection to each one of the VACUTAINER® tubes.

It is another object of the present invention to provide an identification label which can be used to record, identify, and verify the donor's name, date, time, drawer's name, etc.

Still other benefits and advantages of the present invention will become apparent to those of average skill in the art upon a reading and understanding of the following detailed specification.

### BRIEF DESCRIPTION OF THE DRAWINGS

The invention may take physical form in certain parts and arrangement of parts, embodiments of which will be described in detail in the specification, and are illustrated in the accompanying drawings, which form a part hereof and wherein:

FIG. 1 is a perspective view of a tube holder device according to a first embodiment of the present invention with a retaining or mounting strap/band secured around a drawer's hand;

FIG. 2 is a top plan view of a collecting tube holder according to the first embodiment of the present invention;

FIG. 3 is a top plan view of the collecting tube holder according to the first embodiment with collecting tubes secured within a series of retaining sleeves;

FIG. 4 is a side view of the present invention showing a number of collecting tubes within the retaining sleeves according to the first embodiment;

FIG. 5 is a top plan view of the present invention according to a second embodiment including a safety guard;

FIG. 6 is a top plan view of the present invention according to the second embodiment including collecting tubes secured within the retaining sleeves;

FIG. 7 is a side view of the present invention according to the second embodiment;

FIG. 8 is a perspective view of a tube holder device according to the second embodiment of the present invention with a mounting band secured around a donor's arm;

FIG. 9 is a side view of the present invention according to the first embodiment showing a mounting band (i.e. rubber band) attached to a pair of retaining hooks;

FIG. 10 is a front view of the present invention according to the first embodiment showing the collecting tube holder secured to an IV bag and IV pole; and,

FIG. 11 is a side view of the present invention according to the first embodiment showing the collecting tube holder secured to an IV bag and IV pole.

### DETAILED DESCRIPTION OF THE PREFERRED EMBODIMENTS

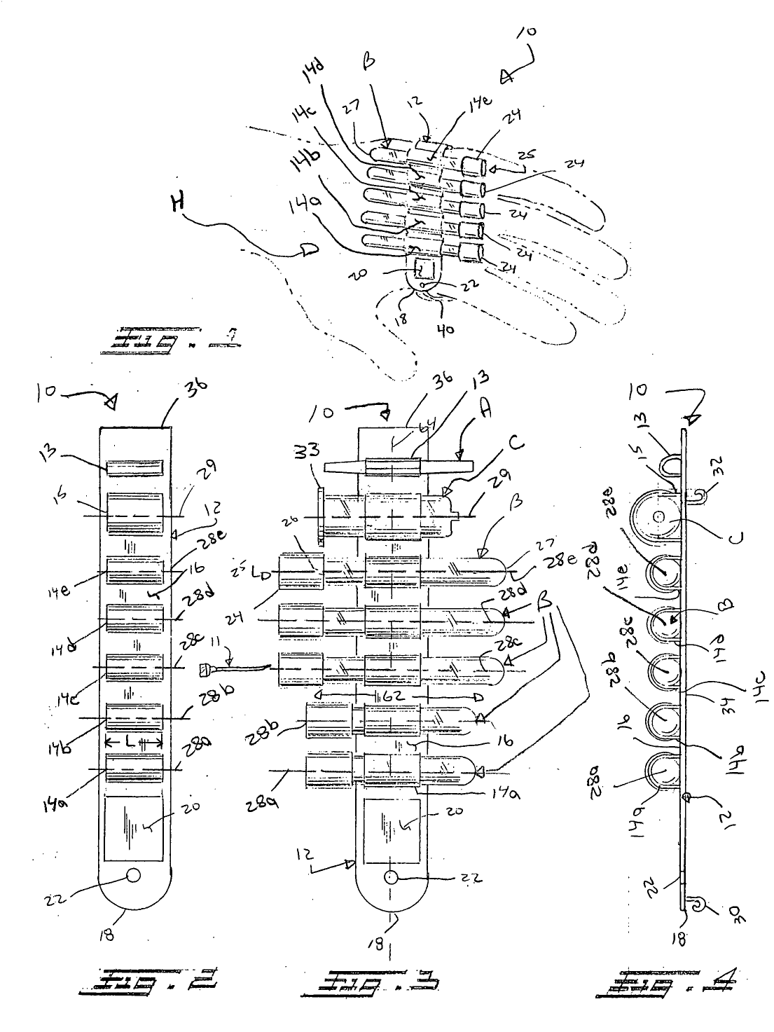

Referring now in greater detail to the drawings, wherein the showings are for the purpose of illustrating preferred embodiments of the invention only, and not for the purpose of limiting the invention, FIGS. 1-4 illustrate a flexible tube holder 10 incorporating an embodiment of the invention. The tube holder 10 can be formed of a durable resilient material such as, but not limited to, a plastic or polymer material. The plastic or polymer material typically includes a material such as, but not limited to, polyvinyl chloride plastic or any other suitable plastic by extrusion, molding, or any suitable plastic forming technique.

Referring to FIG. 1, a perspective view of the tube holder device 10 of the present invention is shown as mounted to a drawer's hand H. Typically, a standard blood drawing situation requires that a series of blood samples be collected from a donor and deposited into separate collection tubes. The sample collection typically is drawn from a location proximal to the donor's inside lower arm, i.e. opposite the elbow. The standard blood drawing tubing or blood drawing system usually consists of two juxtaposed needle elements (i.e. double-ended needle cannula), a first needle or first end (not shown) is used to penetrate a donor's vein (venipuncture) which allows blood to draw from the vein, and a second needle or second end 11 is used to connect to a sample retriever tube C and transfer (or deposit) the drawn blood into a (or a series of) collection tube(s) B. The tube holder 10 device can comprise an elongated flexible body 12 containing a plurality or row of sleeves 13, 14a, 14b, 14c, 14d, 14e, 15 mounted on a top or front side 16 for holding and retaining a needle system tube A, the collection tubes B, and the sample retriever tube C. The sleeves 13, 14a, 14b, 14c, 14d, 14e, 15 can be formed of a durable resilient material such as, but not limited to, a plastic, elastic, elastomeric, or elastic-plastic material. It will be appreciated that all of the tubes B may be held in the device 10 allowing the drawer or technician use of both hands for manipulating the collection tubes B and needle elements. As illustrated in FIG. 3, the sample retriever tube C has a larger diameter and shorter length than the collection tubes B. The needle system tube A can have a smaller diameter relative to the collection tubes B. In the embodiment of FIGS. 1-4, the sleeves 13, 14a, 14b, 14c, 14d, 14e, 15 are generally parallel and can be adapted and sized to hold the typical sizes of needle system tube A, blood collection tubes B, and also one (or more) blood sample retriever tube(s) C. The sleeves 14a, 14b, 14c, 14d, 14e can have a different diameter than sleeves 15 and 13 and can be adapted by, for example, including a ridge(s) or taper along the interior surface of the sleeve (not illustrated). Additionally, the sleeves 13, 14a, 14b, 14c, 14d, 14e, 15 can include a distorted, pinched, restricted, partially collapsed, etc. opening at one or both ends (not illustrated) to facilitate retention of a variety of tube diameters.

Referring to FIG. 2, a top plan view of the tube holder 10 device is shown without the tubes A, B, and C retained in the retaining sleeves 13, 14a, 14b, 14c, 14d, 14e, 15. At one end 18 of the tube holder 10 device, a donor/patient identification area or label 20 can be provided for writing a donor's name, date, time, drawer's name, forms/methods used for patient identification, etc. Also included is a hole 22 for hanging on an IV pole 23 or related apparatus as will be described hereinafter. FIG. 3 is a top plan view of the tube holder 10 device showing a plurality of collection tubes B retained in the retaining sleeves 14a, 14b, 14c, 14d, 14e. Additionally, the sample retriever tube C and needle system tube A can also be retained in retaining sleeves 15 and 13. Typically, the tubes B that are used for collection are the type known in the medical profession as VACUTAINER® tubes. The VACUTAINER® tubes are designed in such a way that a top end 25 is open and a bottom end 27 is closed. The top end 25 includes a rubber stopper 24 around a neck 26 of the tube B. It is to be appreciated that the sleeves 14a, 14b, 14c, 14d, 14e can be sized to allow the tubes B to pass therethrough along a corresponding axis 28a, 28b, 28c, 28d, 28e, but restrict the rubber stoppers 24 proximal to the neck 26 from passing therethrough. Similarly, sleeve 15 can be sized to allow tube C to pass therethrough along a corresponding axis 29, but restrict a flange 33 from passing therethrough. The rubber stopper 24 includes a piercable inner lining, not illustrated, which may be pierced by the depositing needle 11. In light of the fact that varying amounts of blood are required for different types of laboratory analysis, the retaining sleeves 14a, 14b, 14c, 14d, 14e have been designed such that different sized (i.e. different diameters, volumes, lengths, etc.) fluid collection tubes B may be retained by the present invention. The fluid collection tubes B having diameters of between 1.2 and 1.6 cm are generally the most common sizes used for collecting blood samples. However, the tubes can have other sizes which are used, for example, in pediatrics, blood cultures, blood gas collection, etc.

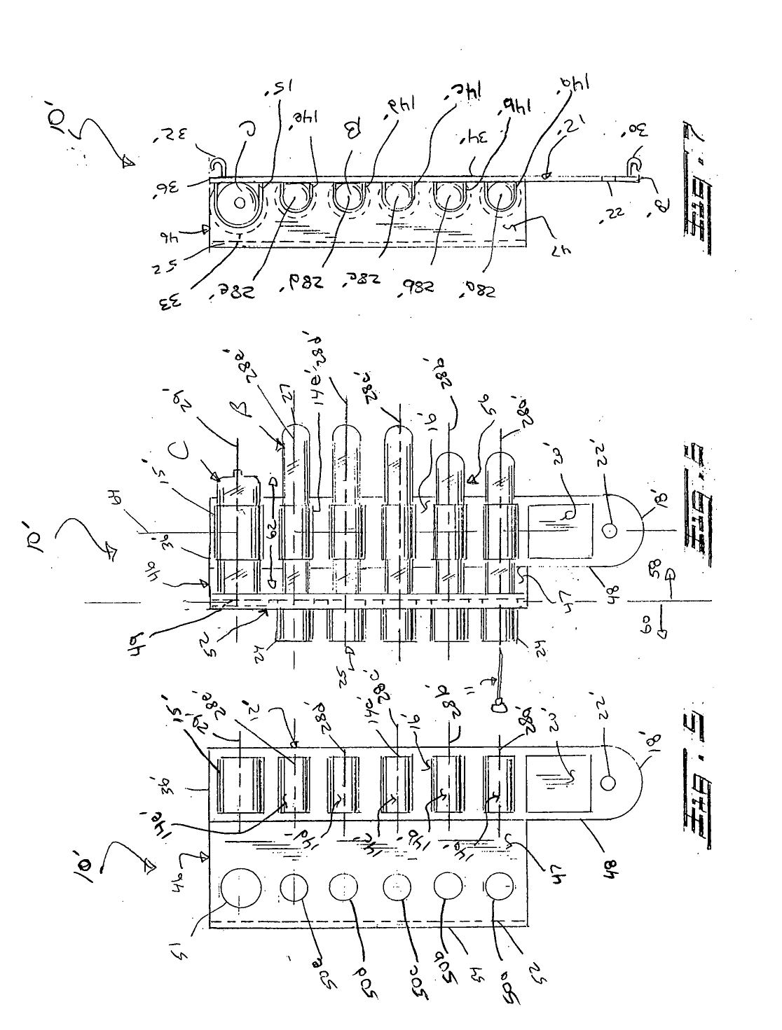

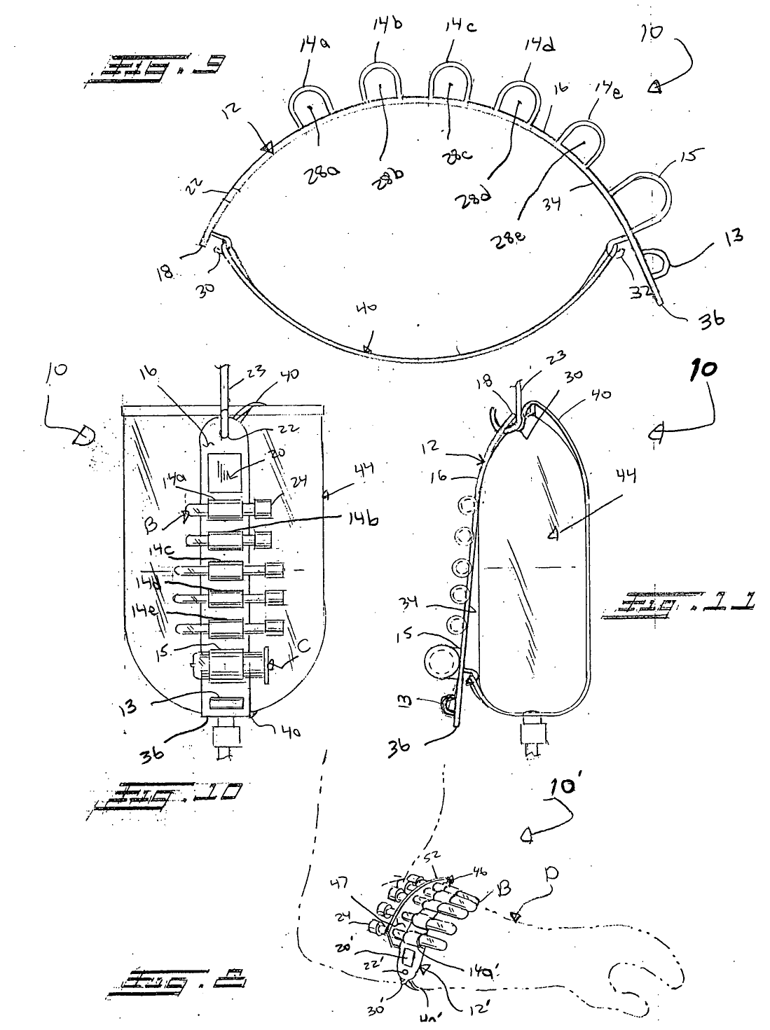

FIG. 4 represents a side view of the tube holder 10 showing the tubes B, C secured in the retaining sleeves 14a, 14b, 14c, 14d, 14e, 15 and also showing a pair of hooks 30, 32 connected to a bottom or back side 34 of the body 12. A first hook 30 can be located at the top end 18 and a second hook 32 can be located proximate to an opposing end 36 of the body 12. The hooks 30, 32 can be used to secure a mounting or securing band 40 (FIG. 9) such that the tube holder 10 and the mounting band 40 can form a flexible and elastic circumferential band which is adapted to surround a technician's hand H (FIG. 1), a donor's arm D (FIG. 8), or an IV bag 44 (FIGS. 10 and 11) which will be described hereinafter. It is to be appreciated that the securing band 40 can be retained by any number of devices. For example, a second hole (not illustrated) can be proximal to end 18 for passing and looping band 40 therethrough. The opposing end of the band can be looped around sleeve 15 (not illustrated) to form a flexible and elastic circumferential band.

A tube holder incorporating another embodiment of the invention is shown in FIGS. 5-8. Like components are identified with like numerals including a primed (′) suffix and new components are illustrated by new numerals. A tube holder 10′ is shown with a protective guard 46. The flexible protective guard 46 or safety shield is shown extending from a side 48 of the tube band body 12′. The protective guard 46 includes a series of openings 50a, 50b, 50c, 50d, 50e configured to receive tubes B and an opening 51 configured to receive the sample retriever tube C. The guard 46 can include an outwardly projecting rim 52 extending along an edge 54 of the protective guard 46. The guard 46 can include soft, flexible top and bottom surfaces 47, 49, respectively. The flexible bottom surface 49 and the projecting rim 52 can be used for the purpose of stopping travel of the needle 11 and preventing the needle 11 from slipping off the guard 46 and striking the drawer's fingers. The flexible guard 46, as shown in FIG. 5, can be flat and coplanar with the band body 12′ or can be folded behind the body 12′, not illustrated, in an inactive position. As shown in FIGS. 6, 7 and 8, the flexible protective guard 46 is folded upward generally perpendicular or orthogonal to the body 12′ whereby a series of tubes B can be inserted first through the openings 50a, 50b, 50c, 50d, 50e and second through the associated retaining sleeves 14a′, 14b′, 14c′, 14d′, 14e′ along respective axes 28a′, 28b′, 28c′, 28d′, 28e′. In this configuration, a bottom section 56 of the tubes B to one side 58 of the protective guard 46 are separated from their respective rubber stoppers 24 at an opposing side 60 of the guard 46. It is to be appreciated that the openings 50a, 50b, 50c, 50d, 50e can be sized to allow the tubes B to pass therethrough but restrict the rubber stoppers 24 from passing therethrough. The protective guard 46 can shield and protect the drawer's fingers from a needle stick injury while taking fluid samples directly from the patient or donor. Similarly, tube C can be inserted first through the opening 51 and second through the associated retaining sleeve 15′ along a respective axis 29′.

It is to be appreciated that the drawer's fingers can be used to slide an individual tube B toward the depositing needle 11 while the fingers remain on side 58 and protected from the needle 11, and while the tubes B remain retained in sleeves 14a′, 14b′, 14c′, 14d′, 14e′. Referring to FIG. 6, it is to be appreciated that each tube B in the tube holder arrangement can in turn be urged toward and away from the depositing needle 11 while, for example, the tube holder 10′ is wrapped around a donor's arm with a band 40′. As described above, the depositing needle 11 pierces the piercable inner liner at the top end 25 of the tube B. As such, the needle 11 remains on one side 60 of the protective guard 46 and the drawer's fingers remain on the other side 58 of the protective guard 46. The retaining sleeves 14a′, 14b′, 14c′, 14d′, 14e′ are of sufficient length L such as to frictionally secure the tubes B while allowing the frictional force to be readily overcome by the drawer to move the tubes B toward and away from the depositing needle 11 while the fingers remain protected from the needle 11 behind guard 46. Similarly, each tube B in the tube holder arrangement can successively, or as needed, be urged toward and away from the depositing needle 11 as each sample is drawn.

Referring to the first embodiment and FIGS. 2-4 and 9, it is to be appreciated that a similar method can be followed in which the drawer's fingers can be used to slide an individual tube B toward the depositing needle 11 while the fingers remain distal to the depositing needle 11 and while the tubes B remain retained in sleeves 14a, 14b, 14c, 14d, 14e. Referring to FIG. 3, it is to be appreciated that each tube B in the tube holder arrangement can in turn be urged toward and away from the depositing needle 11 while, for example, the tube holder 10 is wrapped around a donor's arm with the band 40 (not illustrated). As described above, the depositing needle 11 pierces the piercable inner liner at the top end 25 of the tube B. As such, the needle 11 remains distal to the drawer's fingers while the tubes B are urged toward and away from the needle 11. The retaining sleeves 14a, 14b, 14c, 14d, 14e are of sufficient length L such as to frictionally secure the tubes B while allowing the frictional force to be readily overcome by the drawer to move the tubes B toward and away from the depositing needle 11 while the fingers remain distal to the needle 11. Similarly, each tube B in the tube holder arrangement, after rotating or repositioning the tube holder 10, can successively, or as needed, be urged toward and away from the depositing needle 11 as each sample is drawn.

Additionally, the retaining sleeves 14a, 14b, 14c, 14d, 14e on the top side 16 of the body 12 are of sufficient length L such as to reduce rotational mobility of the tubes B tangential to a central axis 64 of the band body 12. Depending on the dimensions of tubes B, length L can be in the range of 12-88% the length of the tubes B between the neck 26 and the bottom end 27. Restricting rotational mobility minimizes the contact of the fluid collection tubes B with each other during use and transport. As a result of preventing the collection tubes B from contacting each other in this manner, the risk of breaking one or more of them during the blood collection process can be greatly reduced.

In the second embodiment of the present invention, the protective guard 46 adds additional points of contact between the perimeters of openings 50a, 50b, 50c, 50d, 50e and tubes B thereby further restricting rotational movement of the individual collection tubes B. As such, while lateral movement 62 transverse to the central axis 64 of the body 12′ toward and away from the depositing needle 11 is somewhat restricted, the retention forces can be overcome. Rotational movement of the tubes B, tangential to the central axis 64, is restricted by the combined retention forces of the sleeves 14a′, 14b′, 14c′, 14d′, 14e′ and the perimeters of openings 50a, 50b, 50c, 50d, 50e in the guard 46.

Referring to FIG. 9, the mounting band 40 for securing the tube holder 10 to a drawer's hand H, donor's arm D, or IV bag 44 is shown. More particularly, FIG. 9 is a side view of the tube holder 10 with the accompanying mounting band 40 (e.g. nylon strap, rubber band, elastic band or other retention band). Fastening members such as hooks or clips 30, 32 are shown on the bottom side 34 of the body 12 at opposing ends of the tube holder 10. As can be appreciated, other fastening arrangements can be used (e.g. Velcro strap, elastic band sewn to tube holder, hole(s) for looping rubber band, etc.) The resilient plastic material of the band body 12 along with the mounting band 40 allows the tube holder 10 to be secured to an IV bag 44 as shown in FIGS. 10 and 11. The hole 22 in the top end 18 of the holder body 12 can also be used for hanging from the associated IV pole 23. In this orientation, the tube holder 10 can secure the collection tubes B, while at the same time, allows for ready access of the tubes B by the medical personnel. Additionally, the patient information, via the label 20, can be displayed and/or accessed for identification and verification purposes. The identification area 20 can be placed on the back side 34 (not shown) to allow for additional space to document information. It will be appreciated that the hole 22 at the top end 18 of the band body 12 of the IV pole 23 therethrough, can maintain the tube holder 10 in an upright position as the IV bag 44 is depleted. When it becomes necessary to change IV bags 44, it is an easy maneuver to remove the tube holder 10 from the IV pole 23 and used bag 44 by unhooking the body 12 and removing the mounting band 40. The used IV bag 44 can then be removed, and upon resecuring a new IV bag to the IV pole 23, the tube holder 10 can be reengaged as described above.

While considerable emphasis has been placed herein on the structures and configuration of the preferred embodiments of the invention, it will be appreciated that other embodiments, as well as modifications of the embodiments disclosed herein, can be made without departing from the principles of the invention. In this respect, it will be appreciated that the tube holder can be used in various kinds of applications. Likewise, it will be appreciated that a tube holder according to the invention can be of any number of different dimensions. These and other modifications of the embodiments shown will be obvious and suggested to those skilled in the art from the disclosure herein. It is to be distinctly understood that the foregoing descriptive matter is to be interpreted merely as illustrative of the present invention and not as a limitation thereof. Obviously, modifications and alterations will occur to others upon a reading and understanding of this specification. It is intended that the invention be construed as including all such modifications and alterations as fall within the scope of the appended claims or the equivalents thereof.

Having thus described the invention, it is claimed:

1. An apparatus for holding and transporting tubes, comprising:
a tube holder having an elongated body with at least one fluid collection sleeve thereon for receiving and retaining at least one fluid collection tube; and,
at least one sample retriever sleeve thereon for receiving and retaining a sample retriever tube.

2. The apparatus of claim 1, further including at least one needle system sleeve thereon for receiving and retaining a needle system tube.

3. The apparatus of claim 1, wherein said at least one fluid collection sleeve includes a first diameter;
said at least one sample retriever sleeve includes a second diameter; and,
said first diameter less than said second diameter.

4. The apparatus of claim 1, further including a first member and a second member for retaining a securing band therebetween.

5. The apparatus of claim 1, further including a protective guard connected to a side of said holder;
said guard includes at least one fluid collection opening therethrough; and,
said at least one fluid collection opening aligned with said at least one fluid collection sleeve for receiving and retaining said at least one fluid collection tube.

6. The apparatus of claim 5, wherein said guard is hingedly connected to said side of said holder.

7. The apparatus of claim 5, wherein said guard includes an outwardly projecting rim extending along at least one edge.

8. The apparatus of claim 5, wherein said guard is comprised of flexible material.

9. The apparatus of claim 5, wherein said at least first opening is axially aligned with said at least first sleeve when said guard is orthogonal to said holder.

10. The apparatus of claim 1, wherein said body includes a hole therethrough proximal to an end for hanging said tube holder.

11. The apparatus of claim 1, wherein said body includes an identification area for documenting information.

12. An apparatus for holding and transporting tubes, comprising:
a tube holder having at least one fluid collection sleeve thereon for receiving and retaining at least one fluid collection tube; and,
said tube holder including a first member and a second member for retaining a securing band therebetween.

13. The apparatus of claim 12, further including at least one sample retriever sleeve thereon for receiving and retaining a sample retriever tube.

14. The apparatus of claim 13, further including at least one needle system sleeve thereon for receiving and retaining a needle system tube.

15. The apparatus of claim 13, wherein said at least one fluid collection sleeve includes a first diameter;
said at least one sample retriever sleeve includes a second diameter; and,
said first diameter less than said second diameter.

16. The apparatus of claim 12, further including a protective guard connected to a side of said holder;
said guard includes at least one fluid collection opening therethrough; and,
said at least first opening aligned with said at least one fluid collection sleeve for receiving and retaining said at least one fluid collection tube.

17. The apparatus of claim 16, wherein said guard is hingedly connected to said side of said holder.

18. The apparatus of claim 16, wherein said guard includes an outwardly projecting rim extending along at least one edge.

19. The apparatus of claim 16, wherein said guard is comprised of flexible material.

20. The apparatus of claim 16, wherein said at least first opening is axially aligned with said at least first sleeve when said guard is orthogonal to said holder.

21. The apparatus of claim 12, wherein said body includes a hole therethrough proximal to an end for hanging said tube holder.

22. The apparatus of claim 12, wherein said body includes an identification area for documenting information.

23. An apparatus for holding and transporting tubes, comprising:
a tube holder having at least one fluid collection sleeve thereon;
a protective guard connected to a side of said holder;
said guard including at least one fluid collection opening therethrough; and,
said at least one fluid collection opening aligned with said at least one fluid collection sleeve for receiving and retaining at least one fluid collection tube.

24. The apparatus of claim 23, further including at least one sample retriever sleeve thereon for receiving and retaining a sample retriever tube.

25. The apparatus of claim 24, further including at least one needle system sleeve thereon for receiving and retaining a needle system tube.

26. The apparatus of claim 24, wherein said at least one fluid collection sleeve includes a first diameter;
said at least one sample retriever sleeve includes a second diameter; and,
said first diameter less than said second diameter.

27. The apparatus of claim 23, wherein said tube holder includes a first member and a second member for retaining a securing band therebetween.

28. The apparatus of claim 23, wherein said guard is hingedly connected to said side of said holder.

29. The apparatus of claim 23, wherein said guard includes an outwardly projecting rim extending along at least one edge.

30. The apparatus of claim 23, wherein said body includes a hole therethrough proximal to an end for hanging said tube holder.

31. The apparatus of claim 23, wherein said guard is comprised of flexible material.

32. The apparatus of claim 23, wherein said at least one fluid collection opening is axially aligned with said at least one fluid collection sleeve when said guard is orthogonal to said holder.

33. The apparatus of claim 23, wherein said body includes an identification area for documenting information.

34. A method of collecting fluid samples from a donor comprising the steps of:
mounting a tube holder to a donor's arm, said tube holder includes at least one fluid collection sleeve thereon for receiving and retaining at least one fluid collection tube, said tube holder is moveable about the arm;
moving said at least one fluid collection tube toward a depositing needle, said needle is in communication with the fluid sample;
filling said at least one fluid collection tube with the fluid sample; and, moving said at least one fluid collection tube away from said depositing needle.

35. The method of claim 34, further comprising the steps of:
rotating said tube holder about the donor's arm;
moving at least a second fluid collection tube toward said depositing needle;
filling said at least second fluid collection tube with the fluid sample; and,
moving said at least second fluid collection tube away from said depositing needle.

36. The method of claim 35, further comprising the steps of:
dismounting said tube holder from the donor's arm; and,
documenting donor information onto an identification area.
